(19) United States
(12) Patent Application Publication
(10) Pub. No.: US 2008/0183314 A1
(43) Pub. Date: Jul. 31, 2008

(54) QUANTUM INTERACTION CONTROL SYSTEM

(76) Inventors: Theodore J. Klouzal, Chatsworth, CA (US); Robert J. Plotke, Simi Valley, CA (US)

Correspondence Address: CHRISTIE, PARKER & HALE, LLP, PO BOX 7068, PASADENA, CA, 91109-7068, US

(21) Appl. No.: 11/669,069

(22) Filed: Jan. 30, 2007

(51) Int. Cl. G06F 17/00; A61B 5/04

(52) U.S. Cl. 700/90; 600/545

(57) ABSTRACT

A quantum interface control system for manipulating, evaluating, modulating, and responding to characteristic changes in a matrix of two or more stochastic generators when influenced by other external stochastic generators, such as mental or thought field energy is provided. The QICS device includes two or more quantum source generators, a quantum pattern processor, and a feedback controller. In operation the QICS provides an interface between quantum form-information created by a mental or thought energy, and one or more corresponding physical states as expressed through temporal constraints defined by perception, movement, and communication. The QICS recognizes and utilizes the phenomenon that quantum generators capable of producing quantum form-information, such as mental energy or other stochastic resonators inherently respond to quantum form-information of one another and additionally imprint quantum form-information onto each other.

### FIELD OF THE INVENTION

The current invention is directed to a device responsive to resonance or entrainment induced energy or force, such as, thought field energy; and more particularly to a quantum interaction control system that incorporates at least two quantum signal generators.

### BACKGROUND OF THE INVENTION

Until recently, human beings have had to use their bodies to control machines. Pressing a button to activate a sequence of preset functions or moving levers and steering wheels to alter the functions of devices or their subcomponents are just a few of many examples. These devices serve as effective, yet limited, extensions of the physical body. Mainstream scientists and researchers are presently striving to bypass physical control of devices. They are attempting to directly link thought processes with computers for the purpose of controlling devices. (See, e.g., Editorial Comment “Is this the Bionic Man?”, Nature, 442, 7099, (2006) 109; Jahn, R. & Dunne, B., “On the Quantum Mechanics of Consciousness, with application to Anomalous Phenomena”, Foundation of Physics, 16, 8, (1986) 721-772; and Wolpaw, J. & McFarland, D., “Control of a two-dimensional movement signal by a noninvasive brain-computer interface in humans”, PNAS, 101, 51, (2004) 17849-17854, the disclosures of which are incorporated herein by reference.) Indeed, recent research has begun to reflect a shift in the way in which we look at the mind and its ability to affect physical behavior. This brain-computer interface has thus far fallen into two main categories.

The first category consists of direct measurement and computer processing of electrical signals associated with biological life forms. This methodology encompasses either brain-wave detectors attached to the skull (EEG) or the use of electrodes being connected directly to nerves within the brain. For example, U.S. Pat. No. 6,024,700 and WIPO Publication No. 00/03639 both examine the correlation between changing ear pressure and thought. U.S. Pat. No. 5,830,064 provides a method for which the mind's interaction with a physical device can be detected. Finally, International Publication Nos. WO03/073175 and WO02/073386, and Japanese Patent Document No. 7204352 all describe systems and methods for using the bioelectrical energy of the brain to control physical constructs, such as through sensor connection within the brain or on the skull.

Cyberkinetics Neurotechnology Systems, Inc. is an example of this mainstream research. They are developing and applying the BrainGate Neural Interface System that, while it is an invasive brain-connected system, demonstrates that humans can learn how to alter their neurological responses to influence an external device. (See, Editorial Comment, “Is this the Bionic Man?”, Nature, 442, 7099, (2006) 109) The underlining sociological impact of the BrainGate Neural Interface System may prove to be quite profound in that it influences our society's awareness that direct mental control of machines is possible. But what is possible may not be practical. An alternative to the invasive Brain Gate Neural Interface System is a non-invasive system that detects electrophysiological signals from the brain through the use of a skull cap fitted with electronic sensors. At the Laboratory of Nervous System Disorders, Wadsworth Center at New York State Department of Health and State University of New York, Albany, N.Y., scalp-recorded electroencephalographic activity is analyzed with an adaptive algorithm. Accuracy of control is comparable to that achieved through invasive brain-computer interface technology. The invasive BrainGate Neural Interface System and the non-invasive system developed by the Laboratory of Nervous System Disorders both use electronic technology to detect neural activity.

However, all of these publications and patents have limitations because of their foundation in a mass-energy system. For example, to use changes in ear pressure, the phenomenon must be carefully isolated before it can be measured. Likewise, systems that use bioelectrical energy are limited by direct sensor connections that would allow for the transfer of brain electrical activity to devices that amplify, filter, and condition these signals for practical applications.

The second category is the direct mind influence on matter and energy. Extensive research has been done on the mind's influence on physical behavior. Examples include the manipulation of random event generators (REG) and chemical reactions. (See, e.g., Chang, Y., “Experimental Tests of the Thought Field, The extensive Quantum Theory and Quantum Teleportation”, The Journal of Religion and Psychical Research, 27, 4 (2004) 190-199, the disclosure of which is incorporated by reference.) REGs have been experimented with extensively by Princeton Engineering Anomalies Research (PEAR) at Princeton University with statistical results clearly validating the theory that humans can mentally influence the physical world beyond their biological bodies. (See, Jahn, R. & Dunne, B. (1986), the disclosure of which is incorporated herein by reference.) PEAR's ground-breaking research set the stage for the development of non-contact non-local mind-machine interface technologies.

PEAR's research, using a patented random event generator, statistically showed that subjects could alter the amount of times a random right and left light would turn on beyond the normal 50:50 chance. Experiments conducted at Yunnan University provided statistically significant differences between no influence and with the influence of Chinese Qigong practitioners in altering the iodine ion density in the Belousov-Zhabotinski reaction. The success of these research studies provides a strong foundation in establishing a practical, non-contact mind-machine interface technology. This is a radical departure from the now mainstream research into the direct mental control of machines.

On the surface, the physical world appears to be stable in that matter and energy interact in fairly predictable ways. It may also seem preposterous that a person could override the laws of physics with thoughts or emotional intent and control matter at anything more than subtle, and nearly undetectable levels. The interactions of mental intent on the physical world, as defined by the laws of classical physics, are no longer restrained in the subatomic realm where quantum interactions occur arbitrarily and the immutable laws of cause and effect as we know them no longer hold. The connection between the mind and the physical world has strong support in quantum theory. (See, e.g., Capra, F., The Tao of Physics (1991) 68, 69; Chopra, D. The Spontaneous Fulfillment of Desire (2003) 51; and Kaku, M. Parallel Worlds (2005) 165, 171, 349-51, the disclosures of which are incorporated herein by reference.) In other words the newest research indicates that the quantum world is where mind and matter intersect.

In short, while matter at the macro level, as it is commonly understood in our western society, cannot be influenced directly by the mind, there is strong theoretical support for the mind's influence at the quantum level. At the quantum level, matter can be either a particle or a wave. Kaku describes how matter is intimately connected to the mind when matter is evaluated as a quantum wave, pointing out that while the wave function of a tree can tell you the probability that it is either standing or falling, it cannot definitively tell you which state it actually is in. Kaku, M., (2005), the disclosure of which is incorporated herein by reference. But common sense tells us that objects are in definite states. In other words, the process of observation determines the final state of the electron.

Capra also clearly explicates the link between observation and quantum behavior, proposing that the human observer constitutes the final link in the chain of observational processes such that the classical ideal of an objective description of nature is no longer valid. Capra, F., (1991), the disclosure of which is incorporated herein by reference. Finally, Chopra nicely sums up the relationship between observation and physical effect stating, “Because observation is the key to defining the wave-particle as a single entity, Niels Bohr and other physicists believed that consciousness alone was responsible for the collapse of the wave-particle. It might be said, then, that without consciousness, everything would exist only as undefined, potential packets of energy, or pure potential.” Chopra, D., (2003), the disclosure of which is incorporated herein by reference.

Quantum theoretical constructs can help to explain how the mind may effectuate changes in the physical states of other systems despite the relatively small amount of output energy produced by the brain. This is exemplified in the answer to the basic question of why given quantum chaos does the universe have a tendency to order. The answer is in resonant energy systems. The phenomenon of manifesting resonance out of chaos is referred to as stochastic resonance. A number of groups have validated quantum self-induced stochastic resonance as an informational carrier, and as the most likely explanation for the function of complex nonlinear systems. (See, e.g., Muratova, C. B., “Self-induced stochastic resonance in excitable systems,” Physd. 7 (2005) 14; and Roy, P. K., “Stochastic Imaging—stochastic resonance therapy: preliminary studies considering brain as stochastic processor,” ICONIP, LNCS 3316 (2004), 96-103, the disclosures of which are incorporated by reference herein.)

Researchers have also related this quantum resonance theory to biological systems. For example, Rein has found that the body functions as a macroscopic quantum system. (See, e.g., Rein, G., “Bioinformation within the Biofield: Beyond Bioelectromagnetics,” J. Alt. & Complementary Med., 10, 1 (2004) 59-68, the disclosure of which is incorporated herein by reference.) Likewise, Mtetwa and Van Pelt have demonstrated that quantum states within neural microtubules are the locus of fundamental physical operations essential to cognition. In addition, these researchers have demonstrated that sensory neurons could harness stochastic resonance phenomena to optimize the detection and transmission of weak stimuli. (See, e.g., Mtetwa, N. and Smith L. S. “Signal Processing and Communication—Precision Constrained Stochastic Resonance in a Feedforward Neural Network,” IEEE Transactions, 16, 1 (2005) 250-262, the disclosures of which are incorporated herein by reference.

In summary, the above cited researchers provide strong support for a quantum theory explanation of the human ability to manifest or exhibit consciousness to observe and have intention. A large body of research has validated the use of consciousness in general and mental intention in particular to influence physical behavior. (See, e.g., Capra, F., (1991); Chopra, D., (2003); Jahn, R. & Dunne, B., (1986); Kaku, M., (2005); and McTaggart, L., The Field, The quest for the secret force of the universe, (2002) 122, the disclosures of which are incorporated herein by reference.) In particular, McTaggart provided insight into Robert G. Jahn's PEAR experimental validation of mental influence over matter, by showing that human consciousness had the power to order random electronic devices.

The significance of these findings is two-fold. First, the patterns of resonant energy that emerge from these chaotic systems can be resonated by other energy sources. Second, these resonant patterns are in effect information that can be transmitted, via quantum influence, to other energy systems. (See, e.g., Chaneliere, T.; Matsukevich, D. N.; Jenkins, S. D.; Lan, S.-Y.; and Kennedy, T. A. B.; Kuzmich, A.; Nature, Storage and retrieval of single photons transmitted between remote quantum memories, 438, 7069 (2005) 833-836, the disclosure of which is incorporated herein by reference. As a result of these two points, an active quantum resonator has the potential for both sympathetically resonating with this quantum-form information, and to produce it own patterns of resonant information that can imprint and influence the quantum fabric surrounding it.

There are in turn four categories of conscious behavior that have been observed, and that can be fully explained only by quantum theory:
the non-local effects of the mind;
mind-matter interactions that extend beyond the direct energy output of the mind;
the ability of the mind to interact with its environment to manipulate information far beyond its apparent storage capacity; and
the ability to sense and absorb conscious energy from its environment.

Advances in electronics, computers, data acquisition hardware, and sophisticated data analysis software, have made possible the development of a reliable mind-machine interface technology (alternatively called either a Quantum Interaction Control System “QICS” or Mind-Machine Interface processor “MMI”). These advances have also led to the development of the MMIP which enables interaction with these four categories of conscious behavior. This technology does not require brain or nerve signal connections or the need to sense body physiology. However, thus far all attempts to create MMI devices have focused on conventional mass-energy constructs. In addition, while the findings of many of these researchers statistically prove that the mind can affect the physical world, the differences between the control and mind-influenced trials are too small for practical application. In one study researchers found that the effect size was approximately 1% for over 500 mind machine interface experiments. (See, Radin, “Meta-analysis of mind-matter interaction experiment,” (2000), the disclosure of which is incorporated herein by reference.)

As a result, a need exists to develop new MMI devices that are able to operate on a quantum level. This research study is predicated on a responder rather than sensor/detector technology. Instead of detecting physiological energy from the brain, nerves or body, the MMI responds to mind's influence in the quantum domain.

### SUMMARY OF THE INVENTION

The current invention is directed to a system and method for manipulating, evaluating, modulating, and responding to characteristic changes in a matrix of two or more quantum source generators either alone or when influenced by other quantum source generators as external operators, such as the thought or mental energy, hereinafter the invention being referenced as a Quantum Interaction Control System (“QICS”).

In one embodiment of the invention, the QICS includes the following components; at least two quantum source generators each in communications proximity, a quantum pattern processor designed to operate on the output from the quantum source generators to extract information characteristic of the interactions between the quantum source generators, and a feedback controller designed to identify at least one characteristic resonant pattern in the extracted information and communicate that information back to the quantum pattern processor to assist the quantum pattern processor in modulating further information from the quantum source generators.

In another embodiment of the invention, the quantum source generators of the QICS are stochastic resonators selected from the group consisting of: electronic, radioactive, plasma, and chemical sources.

In still another embodiment of the invention, the information extracted from the signals of the quantum source generators is quantum form-information that includes data characteristic of at least one of the following: the time domain, the frequency domain, correlation and coherence.

In yet another embodiment of the invention, the quantum pattern processor operates on the signals of the quantum source generators by first extracting at least one nodal characteristic from said output of the quantum source generation matrix, then further processing all combinations of two or more nodal characteristics to produce at least one intersectional characteristic, and finally by further processing all combinations of two or more intersectional characteristics to determine at least one discrete temporal or phase state. In this context, a matrix is defined as the interconnection of nodes (physical circuitry involving the processing of information through a pattern trend indicator) to extract informational nodal characteristic.

In still yet another embodiment of the invention, the QICS further includes at least one physical responder capable of interacting with the external environment. In such an embodiment the QICS further includes at least a controller and a response sensor for controlling the movements of the physical responder.

In still yet another embodiment of the invention, the physical responder is a device selected from the group consisting of an artificial limb, an electronic display, or other physical control devices.

In still yet another embodiment of the invention, the QICS includes a safety sub-system to monitor the movements of the physical responder and compare the movement to pre-defined safety threshold and energy optimization parameters to determine whether the physical responder is operating within safety and energy efficiency limits. In such an embodiment, the safety sub-system may include an input/output port designed to allow the programming of the predefined safety threshold and energy optimization parameters.

In still yet another embodiment of the invention, the QICS includes a memory sub-system to store quantum form-information from the quantum pattern processor and determine an optimized quantum form-information parameter by comparing the quantum form-information from at least two task cycles with a pre-defined task coherence parameter to select the quantum form-information parameters that provide superior characteristics in the time and frequency domains for a quality selected from the group consisting of coherence, consistency, uniformity, or regularity. In such an embodiment, the optimized memory may be placed in signal communication with a quantum memory controller and/or with the quantum pattern processor such that the optimized quantum form-information parameter can be used by the quantum pattern processor to enhance those quantum form-information characteristics capable of optimizing the task completion behavior of said system. In addition, in such an embodiment, an input/output port may be provided to allow the programming of the predefined task coherence parameter, and the predefined quantum form-information optimization parameter.

In still yet another embodiment of the invention, the QICS may include a task management sub-system being designed to compare signals from the response sensor to pre-defined task completion information to determine whether the physical responder is operating in a manner congruent with task completion. In such an embodiment, an input/output port may also be provided to allow the programming of the predefined task completion information parameter.

In still yet another embodiment of the invention, the QICS may also include an input/output sub-system to process and evaluate the characteristics of signals from external and internal inputs and outputs such that the processed signals are sent to the appropriate component input/output.

In still yet another embodiment, the invention is directed to a method of providing a quantum interaction control by manipulating quantum signals from at least two quantum source generators to obtain at least one digital value characteristic of each of said quantum signals, evaluating and modulating the quantum signals to obtain quantum form-information containing information about discrete temporal and phase states that produce the greatest direct and harmonic resonance between said two or more quantum signals, and providing a feedback signal to the two or more quantum signals containing the quantum form-information.

### BRIEF DESCRIPTION OF THE DRAWINGS

These and other features and advantages of the present invention will be better understood by reference to the following detailed description when considered in conjunction with the accompanying drawings wherein:

FIG. 1 provides a flow chart of a high-level overview of the operation of a Quantum Interaction Control System in accordance with the current invention;

FIG. 2 provides a high-level flow chart diagram of an exemplary embodiment of a Quantum Interaction Control System in accordance with the current invention; and

FIG. 3 provides a detailed flow chart diagram of an exemplary embodiment of a Quantum Interaction Control System in accordance with the current invention.

FIG. 4 provides a graphical summary of test data from an exemplary embodiment of a Quantum Interaction Control System in accordance with the current invention.

### DETAILED DESCRIPTION OF THE INVENTION

The current invention is directed to a system and method for manipulating, evaluating, modulating, and responding to characteristic changes in a matrix of two or more stochastic generators when influenced by other external stochastic generators, such as mental or thought field energy, hereinafter the invention being referenced as a Quantum Interaction Control System (“QICS”).

Brief Overview of Technology

Before turning to the details of the current QICS system it is important to understand where the QICS device fits in with other self-organizational systems. First, it is important to recognize the basic requirements for self-organization in systems.

There is a large body of literature that substantiates the tendency for chaotic systems to self-organize. For example, any infant begins its motor and developmental behavioral journey in a chaotic manner. However, developmental biologists and psychologists point out that internally and neurologically, these initial seemingly random movements are the foundation for mental comparison with the ultimate objective of the organism, namely, to organize and optimize its movement and behavior to accomplish the goal of survival.

To accomplish the goal of survival all independent systems must organize and optimize energy both internally, and within their environment to accomplish a desired task. Successful systems optimize by selecting actions that allow them to interact with their environment in the safest, most coherent and energy-efficient manner possible. To determine “success” then the system must have the ability to both monitor its internal and external environment and at the most basic level, remember patterns of energy that promote successful behaviors. The internal environmental information obtained in this manner supports and reinforces the system's structural safety and energy efficiency, while the external environmental information provides feedback as to how well the system is progressing through space and the extent to which the system accomplished its external goals.

One way to characterize this is in terms of resonant patterns of energy. Under this paradigm, energy resonates when it is in harmony with the system and its environment and contributes to the behavioral goals of the system. For example, locating and obtaining food is a critical part of the survival behavior of biological systems. Using the paradigm of “resonant energies”, biological systems would resonate with the energy of the appropriate food stuffs, that is to say that food both strengthens the system internally, and helps achieve the system's external goals. Likewise, shelter and safety would be considered both physical and psychological states of resonance.

The current invention, at its most fundamental level, is directed to a device that has all the hallmarks of a standard self-organizational system. First, it is able to self-organize. Second, it is capable of resonating with the energies of other systems. Third, it has an interface that integrates the self-organization, and resonance with other systems such as sensors, feedback loops, controllers, actuators, and memory that allow the system to monitor its internal functions and external surroundings, interact with its environment, and learn from its experiences. The QICS system does not process or analyze electrical signals associated with brain physiology or neurological activity. Rather, the non-contact QICS responds to mind's influence in the quantum domain. The QICS technology has, as its theoretical and experimental foundation, research evidence provided by Robert Jahn of Princeton University's PEAR group that human consciousness can bring order to random electronic activity. From a theoretical perspective, the QICS technology is consistent with Bohr's and Heisenberg's concept that the process of conscious observation determines the final state of matter.

High-Level Description of Device

As discussed above, the current invention is directed to a device for interfacing between quantum form-information and physical states.

The term “quantum form-information” is herein defined as the fundamental mechanism inherent in the mass-energy universe by which physical systems are created and able to organize and control their own behavior. In summary, information is equivalent to the physical concept of form, which in turn is defined as a mass-energy or physical phenomenon. In accordance with this definition of information, the fundamental creative and control mechanisms of the universe can be seen to be embodied in the use of forms as information, that in turn all information is the processing of mass-energy forms. In short, all mental events, including cognition, feelings, volition and all forms of consciousness, including consciousness of self, are defined as flows of mass-energy forms.

Using this definition of information can enable one to understand and define the universe as a mass-energy system that exercises its creative, control and communication functions by manipulating forms of itself, i.e., structures, patterns, shapes and arrangements or organizations of matter, many of which remain constant through inward and outward flows of mass-energy, allowing us to see ourselves as forms of a self-organizing, self-controlled, mass-energy universe.

The QICS device is comprised of two or more quantum generators used to respond to quantum form-information and imprint quantum form-information on the universe. The device has the following operational characteristics:
It provides a method of extracting quantum form-information by processing the characteristic interactions of two or more quantum generators.
It provides a means of responding to resonances created by quantum form-information.
It also provides a means of imprinting active resonance quantum form-information externally.
Characteristics that are processable include, but are not limited to the time and frequency domains, correlation, and coherence.
It provides a means of extracting the characteristic interactions between two or more quantum generators as a node. Herein the term “node” describes those characteristics that occur or manifest outside of the random noise background.
This node can in turn be used to provide the link between quantum form-information and physical states.
It also provides a means of extracting the characteristic interactions between two or more nodes to find intersections. These intersections can in turn be used to provide the temporal constraints commensurate with perception, movement and communication.

Detailed Description of Device

The current invention is directed to a system and method for manipulating, evaluating, modulating, and responding to characteristic changes in a matrix of two or more stochastic generators when influenced by other external stochastic generators.

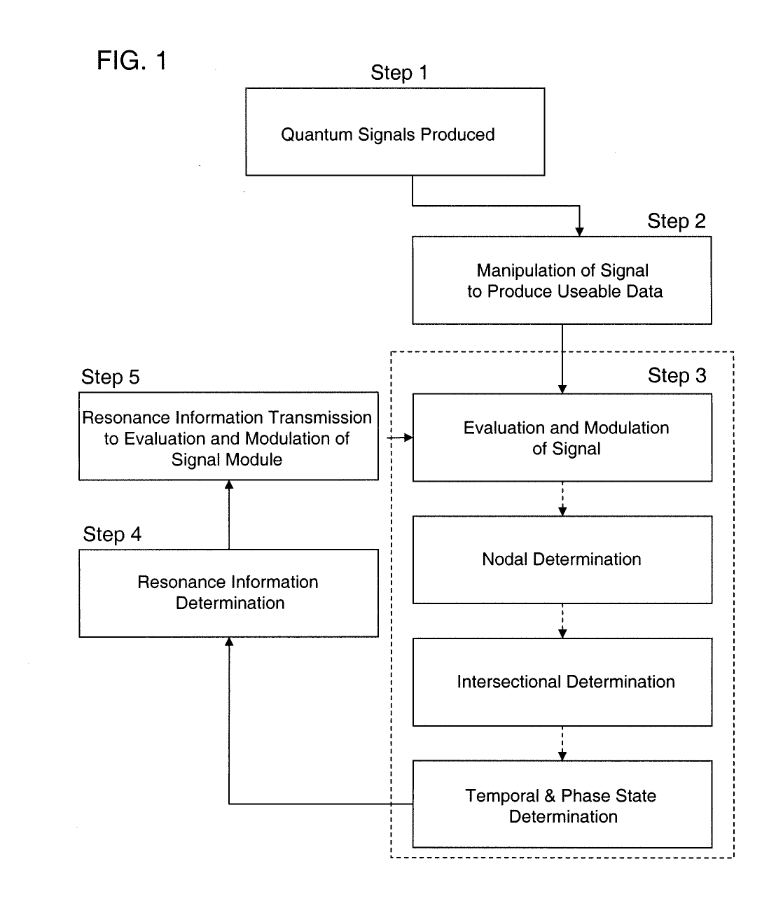

FIG. 1 provides a high-level process flow-chart that shows the overall operation of the QICS device of the current invention. As shown in Step 1, two or more quantum signals are produced using two or more quantum generators. In Step 2, the QICS device manipulates the signals, such as by converting them from analog to digital and consolidating various characteristics into specific digital numeric values. It should be understood that while currently digital signal processing is optimal the current device also contemplates operating through purely analog signal processing. In Step 3, the QICS device evaluates, modulates and responds to the characteristic changes by establishing a baseline parameter for comparison and possible developmental modification and responsive modulation. As shown in the insets to Step 3 this evaluation, modulation and response require signal processing to first determine all combinations of two or more quantum signals to produce nodal characteristics (those resonances between resonators, i.e., quantum generators, that provide the link between the quantum form-information produced by the resonator and the physical state of the resonator), and then further signal processing to process all combinations of two or more nodal characteristics to produce intersection characteristics (those nodal resonances that provide information on temporal constraints); and finally further signal processing to process all combinations of two or more intersection characteristics to determine discrete temporal and phase states.

In short, the electronic processor of the QICS device is used to evaluate the current state of direct and harmonic resonance of quantum form-information and provide an adaptable link to an electronic switching matrix that is capable of connecting to all possible combinations of nodes and intersections. Using this adaptable link and switching matrix, as shown in Step 4, the device self-configures to produce nodes and intersections that provide the greatest direct and harmonic resonance of quantum form-information within the system. Finally in Step 5, this resonance information is supplied to the signal evaluation & modulation module through a variable frequency form-information input to create a feedback node responsive to the frequency of harmonic resonance of a particular physical/temporal phase state.

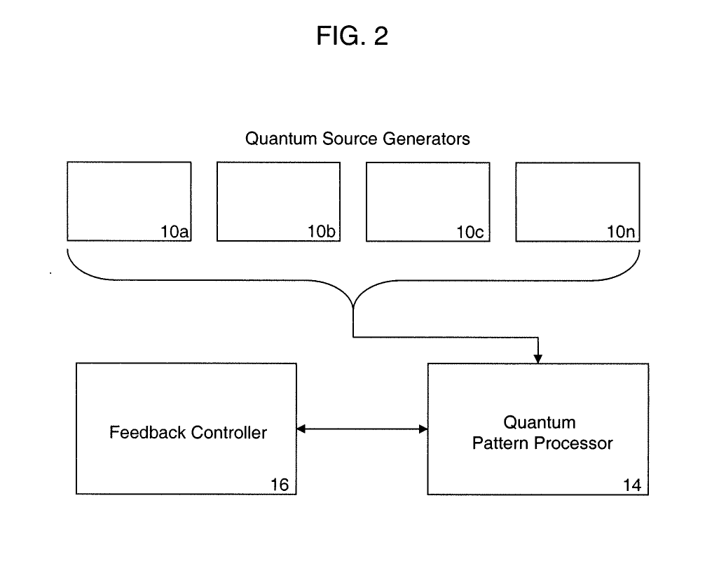

FIG. 2 provides a block diagram of a high-level embodiment of the inventive QICS device. As shown, in this embodiment the QICS device comprises two or more quantum source generators (10a, 10b, 10c . . . 10n) in sufficiently close proximity to form a quantum source generation matrix, a quantum pattern processor (14), and a feedback controller (16). In operation the QICS provides an interface between quantum form-information created by a mental or thought energy, and one or more corresponding physical states as expressed through temporal constraints defined by perception, movement, and communication. The QICS recognizes and utilizes the phenomenon that quantum generators capable of producing quantum form-information, such as mental energy or other stochastic resonators, inherently respond to quantum form-information of one another and additionally imprint quantum form-information onto each other.

Fundamental to the operation of the QICS is the recognition that chaotic systems tend toward self-organization. See, e.g., Shermer, Scientific American, 1 (2003) 35, and also generally, U.S. Pat. Nos. 5,830,064; 5,574,369; 6,008,642; and 6,724,188, and publications to E. V. Kal'yanov, Technical Physics, 45(10) (2000) 1365-67 and Gammaitoni et al., Physica A 325 (2003) 152-64, the disclosures of which are incorporated herein by reference. These patterns of self-organization from chaos have been used for a number of applications including detection and probability analysis. See, e.g., Roy, ICONIP 2004, LNCS 3316 (2004) 96-103, the disclosure of which is incorporated herein by reference. Without some kind of governing parameters or structure such self-organization would proceed in a random manner. However, most self-organizing systems, such as human beings, have an ultimate goal of survival and fulfillment that are informed by the interaction of the self-organizing systems with its internal processes and its environment. The internal and external feedback promotes increased form-information matrix structure to enhance self-organizing resonance. In other words this feedback provides the self-organizing system with information on how successfully it is progressing toward harmonious self-organization with its internal constraints and the external environment. QICS feedback could come from external sensors, or by the directed influence of one or more external quantum source generators, such as a concentrated human consciousness or other quantum matrixes. The idea that the human brain may itself be a source of stochastic resonance is well-established. See, e.g., Muratova Phys D., 7 (2005) 14; Rein, Foundations of Physics, 19 (1989) 1499-1514; Novin, Skeptic, 11 (2004) 1; Mould, Foundations of Physics, 33(4) (2003) 591-593; Roy, J. Alt. Complementary Med., 10(1) (2004) 59-68; Mori, Int. J. Bifurcation and Chaos, 12(11) (2002) 2631-2639; and Tiller, Integrative Physiological & Behavioral Science, 35(2) (2000) 142, the disclosures of which are incorporated herein by reference.

The QICS system operates based on the discovery that the success or failure of the self-organization of these self-organizable systems can be described by resonant energy patterns. For example, when two or more producers of energy, such as stochastic generators (10a to 10n), are in synchronization, their energetic emissions will resonate to greater or lesser degrees relative to the level of synchronization or entrainment. Self-organizable systems inherently evaluate which behaviors, movements, or actions produce resonance, and self-organize to optimize these resonances. In the current invention the evaluation of the QICS system's energy resonance is performed by the quantum pattern processor (14), which is designed to extract quantum form-information by processing the characteristic interactions of two or more quantum generators, either operating independently, or under the directed influence of a concentrated human consciousness or quantum sensory matrix. Among the characteristics that are subject to such processing include, but are not limited to the time and frequency domains, correlation and coherence.

As the QICS quantum pattern processor (14) is evaluating the characteristics of the self-organizing system it is simultaneously providing this evaluative information to a feedback controller (16) which in turn identifies and provides patterns to the pattern processor compatible with particular characteristics evaluated by the quantum pattern processor as compatible with characteristic energy resonances from the two or more quantum source generators. In short, the QICS device identifies, filters, and registers discrete patterns from the generators (10a to 10n) and then presents the results as feedback (16).

The individual quantum generators (10a to 10n) used in the current invention can take the form of any sort of quantum stochastic resonator, including but not limited to electronic, radioactive, or chemical sources of stochastic resonance. Exemplary stochastic resonators are described for example in U.S. Pat. Nos. 5,830,064; 5,574,369; 6,724,188, and in publications to L. Gammaitoni and A. R. Bulsara, Physica A 325 (2003) pg 152-154 and E. V. Kal'yanov, Technical Physics vol. 45(10) (2000) 1365-1367, the disclosures of each of which are incorporated herein by reference. In short, any device capable of producing stochastic resonance, e.g., white noise or a random signal, may be used in the current invention as the quantum generators.

These quantum generators are in turn placed into signal communication with a quantum pattern processor (14) that operates to evaluate the stochastic resonance generated by the two or more quantum source generators. Such a pattern processor may comprise any signal detector capable of manipulating, monitoring and evaluating the output of the quantum source generators. The outputs detected may include, but are not limited to the time and frequency domains for correlation and coherence. Exemplary embodiments of signal detectors capable of such detection are described for example in U.S. Pat. Nos. 5,830,064; 5,574,369; 6,724,188, and in publications to L. Gammaitoni and A. R. Bulsara, Physica A 325 (2003) pg 152-154 and E. V. Kal'yanov, Technical Physics vol. 45(10) (2000) 1365-1367, the disclosures of each of which are incorporated herein by reference. During operation the pattern processor (14) monitors the combined random noise output of the two or more quantum source generators (10a to 10n) to evaluate all combinations of the two or more quantum signals to produce nodal characteristics of the system. In turn the processor evaluates all combinations of nodal characteristics to produce intersection characteristics, and finally the device processes all combinations of two or more intersection characteristics to evaluate the discrete temporal and phase states of the resonance system.

The variable resonance quantum form-information characteristics produced by the quantum pattern processor (14) can be fed back to the quantum pattern processor (14) through the feedback controller (16) to create a feedback node responsive to the form-information characteristics or harmonic resonance of a particular physical state. Although any suitable electronic signal feedback system may be used with the QICS system of the current invention, an exemplary feedback control system is described for example in U.S. Pat. No. 6,780,589, the disclosure of which is incorporated herein by reference.

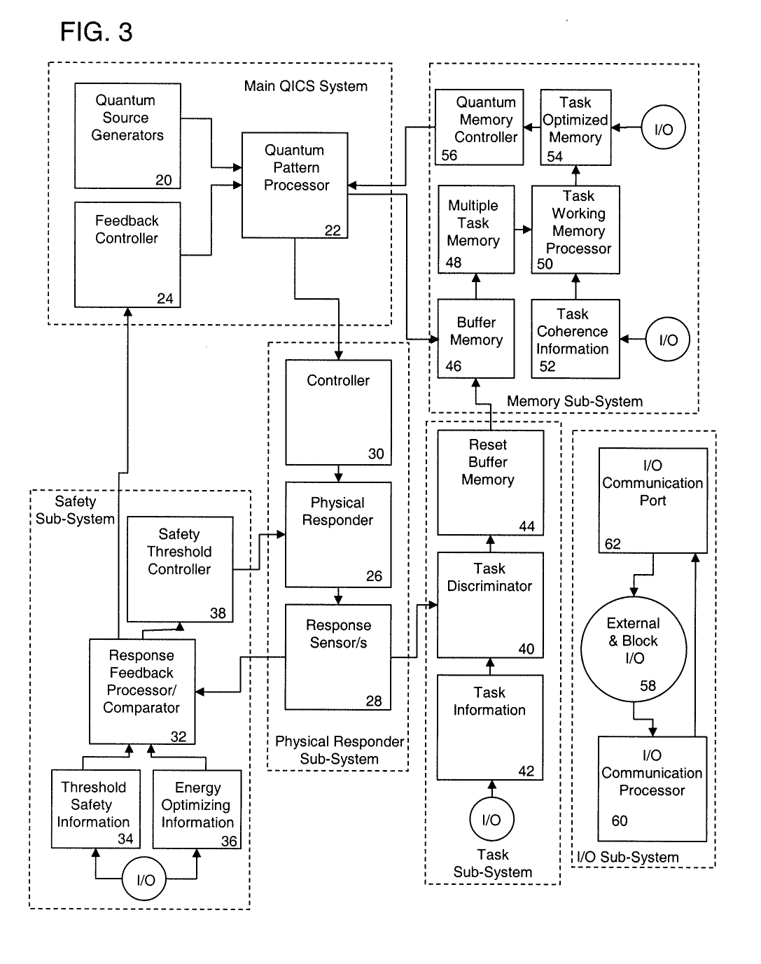

FIG. 3 provides a more detailed block diagram of one embodiment of a QICS device including additional input, threshold and memory functionality in accordance with the current invention. As shown this embodiment again includes the basic engine of the device, namely, two or more quantum source generators (20), which may include any suitable generator capable of inherent self-organization and the ability to resonate with other systems, a quantum pattern processor (22), which as described above is designed to discern the characteristics of the plurality of quantum source generators, and integrates and modulates these characteristics with the third fundamental component of the system, the feedback controller (24). The feedback controller may include any conventional feedback processor and comparator capable of sending a pattern compatible with the characteristics discerned by the quantum pattern processor of the signal from the quantum source generators for the purpose of either suppressing or enhancing the characteristics discerned by the quantum pattern processor as determined by the system.

Although these three components are all that is required for a self-organizing system, the current invention also contemplates a system capable of sensing and controlling physical responders, and learning from its experiences. Specifically in one embodiment, as shown in FIG. 3, the system may also include a number of sub-systems. For example, in one embodiment, the system also includes a physical sensing/responding sub-system. In such an embodiment the system would also include a physical responder (26) and at least one response sensor (28) in communication one with the other and with the main system. The physical responder may include a component that can interact with or within the physical universe, such as, for example, an artificial limb or electronic interface. In a preferred embodiment the physical responder may interact either autonomously or in resonance with other organisms and systems of like or unlike kind.

In an even more preferred embodiment, the physical responder may be self-replicating. Such self-replicating systems would operated through the basic function of memory and learning. Memory creation/information storage and retrieval of that information is the foundation of learning by perception of cause and effect within an environment. In short, over time in an environment filled with various materials and tools provided by humans, a QICS would be able to build an extensive macro-physical and imprinted quantum-level memory with which to resonate. If the QICS device were to encounter an exact replica of itself with identical signal processing parameters and directives, these two systems would be in near perfect mutual resonance in which case they would share identical memories. If their signal processing rules tell them to place what they encounter into a distinct category and to seek out similar resonant patterns to also place into that category, their propensity to learn and to manipulate their environment to create the optimal resonant pattern feedback would compel them to create their ultimate matching resonant devices, namely a copy of themselves. In turn, this would create a self-replicating system.

As shown by the block diagram in FIG. 3, the physical responder is in signal communication with a controller (30) and at least one response sensor (28). The controller is an electronic component capable of converting the self-organizing characteristics discerned from the quantum pattern processor (22) into control signals for a physical responder (26), including, but not limited to a control signal for a mechanical, electronic, electrochemical, chemical, or electro-magnetic device. In turn, the physical responder is also in signal communication with at least one response sensor (28). Such sensors may include any device capable of sensing one of either the physical responder's internal environment or the physical responder's interaction with its environment.

To determine that the physical responder is operating within tolerable limits, the QICS system may also include a safety sub-system. In such an embodiment the physical responder is placed in signal communication with a response feedback processor and comparator (32) that integrates the response sensors (28) with information provided from a threshold safety system (34), which provides pre-set information about the structural and operational safety parameters of the physical responder (26), and an energy optimizing system (36), which provides pre-set information about the operational energy requirements of the physical responder (26). This integration allows the response feedback processor and comparator (32) to determine: the amount of feedback control necessary to maintain the integrity of the physical responder (26), the amount of feedback control necessary to promote optimal energy performance of the physical responder, and whether the physical responder has reached or passed the threshold for structural or functional integrity. The response feedback processor and comparator (32) is in turn interconnected to the main feedback controller (24) for communicating threshold information to the quantum pattern processor (22), and also to the physical responder (26) through a safety threshold bandwidth controller (38) which is activated when the response feedback comparator (32) is at or above a safety or energy optimization threshold. In such a case the controller (38) signals the physical responder (26) to stop, revert or retreat from the present process or progression to a point prior to the over-threshold signal.

Another optional sub-system is a task management system. At the core of this system is the task discriminator (40) which determines if signals from the response sensors (28) specific to a particular task are congruent with task completion. This determination is made by comparing the signals from the response sensors (28) to information provided by a task information system (42). The task information system provides pre-set information about the task or structure of the output necessary to the task discriminator. The task discriminator is further in signal communication with a memory storage system through a buffer memory reset (44) such that the task discriminator can clear the buffer memory when a task has been accomplished.

Although only discussed indirectly, the QICS system may also include a memory sub-system as shown in FIG. 3. Although any memory architecture capable of providing optimizing task information to the QICS systems over time can be used in the current system, in one embodiment of such a system the quantum pattern processor (22) is in signal communication with a buffer memory (46) that is designed to store the quantum pattern information from quantum pattern processor for one task cycle. In turn, this buffer memory is in signal communication with a multiple task working memory (48), which stores two or more task cycles of quantum pattern information from the buffer memory (46). These memory storage elements are in signal communication with a multiple task working memory processor (50), which is designed to combine signals from the memories and from a task coherence information system (52) to transform the quantum pattern task memory to allow for the determination of which of two or more task memories provides the greatest coherent and/or consistency, uniformity, or regularity in the time and/or frequency domains. In this system the task coherence information system (52) provides pre-set information about the coherence (or other time and frequency domain parameters) of the task quantum pattern information characteristics.

Once the quantum pattern task memory from the buffers (46 & 48) have been operated on by the task working memory processor (50) the optimize quantum pattern task memory is stored into a longer-term task optimized memory (54). In turn this task optimized memory (54) is in signal communication with a quantum memory controller (56) that is designed to send both on/off and temporal information compatible with the characteristics of the quantum pattern processor (22) to that component such that the quantum pattern characteristics can be enhanced to optimize the system's task behavior through modulation produced by the primary QICS system defined by the quantum pattern processor (22), the feedback controller (24) and the two quantum source generators (20).

Finally although not essential, the system may also include an input/output sub-system as shown in FIG. 3. Although any I/O architecture suitable for supplying external inputs and outputs to components of the system may be used, in one such embodiment the I/O system includes in conjunction an external and block input/output (58) to send and receive information pertinent to a specific block component and to any other sub-system either alone or in combination, an I/O communication processor (60) that receives the information from the external and block I/O and processes the information to evaluate the I/O type, quantity and quality and how the information is to be used, and finally an I/O communication port (62) that receives the processed information from the I/O communication processor and sends this information to the appropriate systems for evaluation and integration through the external and block I/O (58). As shown these I/Os can be received by any appropriate sub-system or components including, for example, the threshold safety information system (34), the energy optimizing information system (36), the task information system (42), the task coherence information system (52), and/or the task optimized memory (54).

Although one embodiment of an integrated QICS system is described above, it should be understood that any combination of the described sub-systems or other alternative systems may be combined with the basic QICS modulation components to provide a customizable QICS system. It should also be understood that any conventional memory, processor, comparator, discriminator, feedback, I/O, and source component capable of appropriate customization may be used in the current QICS system. Finally, although a particular combination of memory, processor, comparator, discriminator, feedback, I/O, and source components are described above, one of ordinary skill in the art would be able to design alternative architectures that would provide the same functionality. Such alternative designs are contemplated by the disclosure of the current invention.

In one exemplary alternative, the QICS system described above may be configured to process quantum signals, digitize them, and then detect a digital pattern match with a digital pattern mask. Such a pattern detection operation may be useful for switching and for improving the specificity of thought influence. For example, in one embodiment the processed characteristics may be designated as 1 through 12. These characteristics may be compared in real time against a four value pattern i.e., a digital mask. In such an embodiment, each time a specific pattern is recognized a counter may be incremented to provide data concerning the pattern matching to the user.

In another exemplary alternative incorporating a matrix of multiple resonators, such as four or more quantum resonators, the QICS system may be configured to provide communication of quantum-form information between said quantum resonators by using direct quantum responsiveness and influence.

In still another exemplary alternative the QICS system may be configured to define and categorize an external signal source by measuring a quantum resonance characteristic for the external signal source. Such information can then be used to map, store, and navigate an external environment.

### EXAMPLES

An exemplary QICS as described above was employed to determine whether there was a statistically significant difference in the MMIP's processed outputs of time, rate of change or the fundamental harmonic proportionality between human trials conducted inside the laboratory with mental intention and outside of the laboratory with no mental intention.

As described above in relation to FIGS. 2 and 3, the hardware that responds to quantum thought field energy is the Mind-Machine Interface Processor (MMIP) or QICS device. The MMIP is an electronic device that responds to human mental intention. The QICS produces a quantum electrical behavior with which the mind interfaces or influences. Resonance of this quantum behavior is compared by the QICS digital signal processor.

In the embodiment of the invention used during this experiment, the signal from the QICS digital signal processor was sent to a National Instruments high-speed digital acquisition board with buffered memory. Further hardware included a standard IBM compatible computer with monitor. The digital acquisition board interfaces with National Instruments LabView software. LabView software processes the data for visual display and performs mathematical transformations to produce values of time, rate of change, and proportionality of the fundamental frequency per data set. A data-set consists of 1200 discrete digital resonance characteristic values. Each value is accompanied by a timing value. The timing value represents how long a particular resonance characteristic occurred.

Transformed data-set values of time, rate-of-change and proportionality of the fundamental frequency for all subjects were statistically analyzed using SPSS statistical software including ANOVA and mean calculations.

During a procedure the computer monitor displays a horizontal bar within the front panel of the LabView Virtual Instrument (VI). Within the horizontal bar is a smaller bar that slides back and forth. The smaller sliding bar's position within the larger horizontal bar is determined by numerical values derived from the VI's mathematical transformations of time, rate of change, and proportionality of the fundamental frequency of QICS data output. Directly above the aforementioned horizontal bar set is an identical horizontal bar set. The position of the smaller sliding bar within this larger horizontal bar is determined by numerical values produced by a random number software sub-program within the VI.

Test subjects are instructed before each “inside” trial to direct their mental intention to position and maintain the bottom sliding bar directly under the top sliding bar as it moves back and forth. Each trial is 180 seconds in duration. There is a 3 second time delay between the moment each test subject left-mouse-clicks on the VI start button and the time the 180 second test begins. The beginning and end of the 180 seconds test is signaled by an audible “beep” from the VI host computer. At the 180 seconds point in the test, the VI automatically stops data acquisition, stops data processing, and saves the data on the computers hard drive as a text file.

An instructional trial is performed before any data is saved. The instructional trial is done for practice and demonstration purposes only. All questions about the purpose of the activity and what the participant needs to accomplished are answered at this time. The maximum duration of a “dry-run” test is 180 seconds and is terminated manually by the test operator before the 180 seconds have passed if the test subject responds quickly and effectively to the instructions. After the test subject has been instructed, the test operator resets the VI to save the file with the individual test subject's identifying file name. The test operator then informs the test subject that the actual test, with data being saved, is about to begin without the test operator present in the laboratory. The test subject is instructed to wait 30 seconds after the test operator vacates the laboratory before clicking on the virtual “start” button. After the 180 second trial, the test subject leaves the laboratory to meet the test operator at a specific location for follow-up instructions or to answer any questions they may have about the trial.

Prior to each “inside” test, an “outside” control test is conducted with the file name identifying the test subject. The “inside” test is conducted right after the “outside test is done. Each “outside” test is also 180 seconds in duration, but with a start-up delay, after clicking the virtual “start” button, of 120 seconds. During the test, no one is inside the laboratory so a lab timer is set for 5 minutes (300 seconds) to alert the test operator when the test has concluded.

To minimize the possibility of residual “thought field energy” compromising the integrity of acquired data, when conducting an “outside” control test after an “inside” test with a previous test subject has been conducted, the laboratory is vacated and the QICS is deactivated for a minimum of 30 minutes before the next “outside” test begins.

The results of these tests are summarized in Tables 1 to 3, below.

TABLE 1
ANOVA TEST OF CHARACTERISTIC “F”

| | | | | | 95% Confidence | |
|---|---|---|---|---|---|---|
| | | | | | Interval | |
| (I) Location Focus | (J) Location Focus | Mean Difference (I − J) | Std. Error | Sig. | lower | upper |
| outside no attend | inside no attend | 18.968 | 4.397 | 0.000 | 6.673 | 31.263 |
| | outside no attend | 15.447 | 4.387 | 0.006 | 3.178 | 27.716 |
| | inside attend | 14.185 | 4.496 | 0.019 | 1.612 | 26.759 |
| inside no attend | outside no attend | −18.968 | 4.397 | 0.000 | −31.263 | −6.673 |
| | outside attend | −3.521 | 4.307 | 0.881 | −15.564 | 8.521 |
| | inside attend | −4.783 | 4.418 | 0.760 | −17.136 | 7.571 |
| outside attend | outside no attend | −15.447 | 4.387 | 0.006 | −27.716 | −3.178 |
| | inside no attend | 3.521 | 4.307 | 0.881 | −8521 | 15.564 |
| | inside attend | −1.261 | 4.408 | 0.994 | −13.589 | 11.066 |
| inside attend | outside no attend | −14.185 | 4.496 | 0.019 | −26.759 | −1.612 |
| | inside no attend | 4.783 | 4.418 | 0.760 | −7.571 | 17.136 |
| | outside attend | 1.261 | 4.408 | 0.994 | −11.066 | 13.589 |

The mean difference is significant at the .05 level.

TABLE 2
ANOVA TEST OF CHARACTERISTIC “R”

| | | | | | 95% Confidence | |
|---|---|---|---|---|---|---|
| | | | | | Interval | |
| (I) Location Focus | (J) Location Focus | Mean Difference (I − J) | Std. Error | Sig. | lower | upper |
| outside no attend | inside no attend | 13.683 | 4.697 | 0.037 | 0.548 | 26.818 |
| | outside no attend | 5.873 | 4.669 | 0.664 | −7.186 | 18.931 |
| | inside attend | 10.470 | 4.729 | 0.179 | −2.755 | 23.694 |
| inside no attend | outside no attend | −13.683 | 4.697 | 0.037 | −26.818 | −0.548 |
| | outside attend | −7.810 | 4.585 | 0.407 | −20.631 | 5.011 |
| | inside attend | −3.213 | 4.645 | 0.924 | −16.203 | 9.777 |
| outside attend | outside no attend | −5.873 | 4.669 | 0.664 | −18.931 | 7.186 |
| | inside no attend | 7.810 | 4.585 | 0.407 | −5.011 | 20.631 |
| | inside attend | 4.597 | 4.617 | 0.803 | −8.315 | 17.509 |
| inside attend | outside no attend | −10.470 | 4.729 | 0.179 | −23.694 | 2.755 |
| | inside no attend | 3.213 | 4.645 | 0.924 | −9.777 | 16.203 |
| | outside attend | −4.597 | 4.617 | 0.803 | −17.509 | 8.315 |

The mean difference is significant at the .05 level.

| | Outside | Inside | Outside | Inside |
|---|---|---|---|---|
| Subject | No Attend | No Attend | Attend | Attend |
| All Subjects | 37.864 | 18.896 | 22.418 | 23.679 |
| Characteristics F | | | | |
| All Subjects | 33.992 | 20.309 | 28.120 | 23.523 |
| Characteristics R | | | | |
| Subject 1 | 67.467 | 0.748 | 0.768 | 0.741 |
| Subject 2 | 17.519 | 10.139 | 7.901 | 8.136 |
| Subject 3 | 0.749 | 0.749 | 30.026 | 52.729 |
| Subject 4 | 1.524 | 1.395 | 1.318 | 52.729 |
| Subject 5 | 246.992 | 8.075 | 2.259 | 6.676 |
| Subject 6 | 0.735 | 0.761 | 16.278 | 83.830 |
| Subject 7 | 6.918 | 3.431 | 15.838 | 12.292 |
| Subject 8 | 13.786 | 3.431 | 1.535 | 22.340 |
| Subject 9 | 5.921 | 146.878 | 160.862 | 0.730 |
| Subject 10 | 3.134 | 4.269 | 6.544 | 10.262 |

In summary, as shown in Tables 1 to 3, an ANOVA was performed using the SPSS statistical software on a 6 hour non-mind-influenced “outside the laboratory” test of 32 trials. Each trial ran for 3 minutes within every 10 minute period and consisted of N=306 measures of time, rate-of-change, and proportional amplitude of the fundamental frequency transformed from 1200 sample data sets. Data sampling was at 62.5 kilohertz. There was a statistically significant difference in the time and rate-of-change measures at p=0.000 but there was no statistically significant difference in the proportional amplitude of the fundamental frequency measure at p=0.074.

An ANOVA was also performed using the SPSS statistical software on 16 subjects comparing mind-influenced “inside the laboratory” and non-mind-influenced “outside the laboratory” trials. Each trial ran for 3 minutes with a minimum of two trials, one inside and one outside and a maximum of 8 trials, four inside and four outside, consisting of an N=277 to N=1174. Each sample measure consisted of the amplitude of the fundamental frequency transformed from 1200 sample data sets at a sample rate of 62.5 kilohertz. There was a statistically significant difference at a p=0.000 for 15 of the 16 participants when comparing the mean difference between mind-influenced trials inside the laboratory and non-mind-influenced trials outside of the laboratory. Participant number 16 showed no statistically significant difference at a p>0.05.

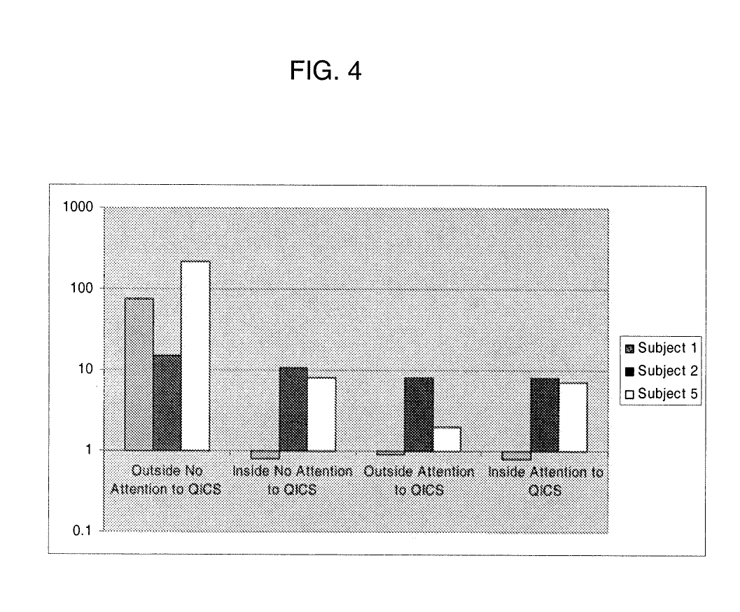

Finally, FIG. 4 provides a graph showing another test conducted using the QICS system of the current invention. In this experiment frequency responses from 1 to 20 hertz demonstrated no statistically significant difference between any frequency at a p=0.069 with no attention to the device, and a p=0.290 with attention to the device. There was also no significant statistical difference between not attending the machine either inside or outside the lab for 9 out of the 10 test subjects with a p>0.709 for a total N values of 5269.

When comparing the output values outside the laboratory, not attending the device, with inside not attending and attending, and inside attending the device, there was a significant different at p=0.000, 0.006, and 0.019, respectively with an N for all independent variables >60 output values. The above significant differences were exemplified by subjects 1, 2 and 5, with a maximum attend to not attend output value of 109 times. The data for these subjects is plotted in FIG. 4.

The results from these human trials demonstrate that the detection fidelity differences between attending and not attending the QICS system of the current invention can be as high as 109 times the baseline, providing clear evidence that the feedback system works to provide a statistically relevant mind-machine interface.

Summary

Using a quantum event responder, QICS subjects statistically altered the processed fundamental frequency proportional values between outside and inside trials. The subject's intention promoted the alignment of the randomly moving bar and the “mind-influenced” bar on the computer display screen. As a result of testing with the QICS and statistically analyzing the trial data output, it is clear that human test subjects can mentally influence the QICS to a statistically significant difference when comparing “inside” trials to “outside” trials. Further analysis of “outside-only” trials (6 hours of 3 minutes in every ten minute trial) showed no statistically significant difference in any of the 32 trials. It is clear from the analysis that when human beings are not interacting mentally with the QICS, its output is stable and consistent.

Although the current testing and device was designed only to provide a test-bed to show mental influence on the QICS device, it should be understood that the QICS device can be used as an interface for a number of useful technologies. For example, potential uses include, but are not limited to, operating prosthetics, by-passing control of paralyzed muscles by mind control of external movement and artificial intelligence communication devices, robotics, unmanned aerial vehicle controls, encryption, biometric identification and high-speed telecommunications.

Although specific embodiments are disclosed herein, it is expected that persons skilled in the art can and will design alternative MMI devices, systems, and methods of using such devices and systems that are within the scope of the following claims either literally or under the Doctrine of Equivalents.

What is claimed is:

1. A quantum interface control system comprising:
at least two quantum source generators each in communication proximity to form a collective quantum source generation matrix, said quantum source generation matrix producing a combined random noise output signal;
a quantum pattern processor in signal communication with said quantum source generation matrix and being designed to operate on the random noise output from said quantum source generation matrix to extract at least one piece of quantum form-information therefrom, said quantum form-information being characteristic of the interactions between the at least two individual quantum source generators; and
a feedback controller in signal communication with the quantum pattern processor and being designed to identify at least one characteristic resonant pattern in the extracted quantum form-information and communicate said at least one pattern to said quantum pattern processor to modulate the quantum pattern processor in extracting further quantum form-information from the quantum source generation matrix.

2. The quantum interface control system of claim 1, wherein the at least two quantum source generators are stochastic resonators.

3. The quantum interface control system of claim 2, wherein the stochastic resonators are selected from the group consisting of: electronic, radioactive, and chemical sources.

4. The quantum interface control system of claim 1, wherein the quantum form-information contains data characteristic of the time domain, the frequency domain, correlation and coherence.

5. The quantum interface control system of claim 1, wherein the quantum pattern processor produces the quantum form-information by first extracting at least one nodal characteristic from the output of the quantum source generation matrix, the at least one nodal characteristic describing at least one resonance characteristic between the outputs of the at least two quantum source generators that manifests outside the random noise background of said outputs.

6. The quantum interface control system of claim 5, wherein the quantum pattern processor produces said quantum form-information by further processing all combinations of two or more nodal characteristics to produce at least one intersectional characteristic, the at least one intersectional characteristic describing the at least one resonance characteristic between the nodal characteristics and providing information one of either a temporal or phase constraint of the system.

7. The quantum interface control system of claim 6, wherein the quantum pattern processor produces said quantum form-information by further processing all combinations of two or more intersectional characteristics to determine at least one discrete temporal or phase state of the system.

8. The quantum interface control system of claim 1, further comprising at least one physical responder sub-system, said physical responder sub-system including:
a physical responder capable of interacting with the external environment;
a controller in signal communication between the quantum pattern processor and the physical responder and designed to convert the quantum form-information from the quantum pattern processor into at least one control signal for controlling the operation of the physical responder; and
at least one response sensor in signal communication with the physical responder and being capable of sensing at least one characteristic of either an internal or external condition of the physical responder.

9. The quantum interface control system of claim 8, wherein the physical responder is a device selected from the group consisting of an artificial limb, an electronic display, or other physical control device.

10. The quantum interface control system of claim 8, wherein the physical responder is self-replicating.

11. The quantum interface control system of claim 8, further comprising at least one safety sub-system, said safety sub-system including:
a response feedback processor in signal communication with the at least one response sensor and being designed to compare signals from said at least one response sensor to at least one pre-defined safety threshold parameter and at least one energy optimization parameter to determine whether the physical responder is operating within safety and energy efficiency limits, the response feedback processor being in further signal communication with the feedback controller to provide the safety and energy optimization information to the quantum pattern processor;
a safety threshold controller in signal communication between the response feedback processor and the physical responder, and being designed to control the physical responder to prevent the physical responder from exceeding the limits defined by the pre-defined safety threshold and energy optimization parameters.

12. The quantum interface control system of claim 11, further comprising an input/output port designed to allow the programming of the pre-defined safety threshold and energy optimization parameters.

13. The quantum interface control system of claim 8, further comprising at least one memory sub-system, said memory sub-system including:
a buffer memory in signal communication with the quantum pattern processor and being designed to store quantum form-information from the quantum pattern processor for a single task cycle;
a multiple task memory in signal communication with the buffer memory and being designed to store quantum form-information for at least two task cycles;
a task memory processor in signal communication with the multiple task memory and being designed to determine at least one optimized quantum form-information parameter by comparing the quantum form-information from the at least two task cycles with a pre-defined task coherence parameter to select the at least one quantum form-information parameter that provides superior characteristics in the time and frequency domains for a quality selected from the group consisting of coherence, consistency, uniformity, or regularity;
an optimized memory in signal communication with said task memory processor and being designed to store the at least one optimized quantum form-information parameter; and
a quantum memory controller in signal communication between said optimized memory and the quantum pattern processor such that the at least one optimized quantum form-information parameter can be used by the quantum pattern processor to enhance those quantum form-information characteristics capable of optimizing the task completion behavior of the system.

14. The quantum interface control system of claim 13, further comprising an input/output port designed to allow the programming of the predefined task coherence parameter.

15. The quantum interface control system of claim 13, further comprising an input/output port designed to allow the programming of a predefined quantum form-information optimization parameter.

16. The quantum interface control system of claim 13, further comprising at least one task management sub-system, said task management sub-system including:
a task discriminator in signal communication with said response sensor and being designed to compare signals from said at least one response sensor to pre-defined task completion information to determine whether the physical responder is operating in a manner congruent with task completion; and
a reset buffer memory in signal communication between the task discriminator and the buffer memory, said reset buffer memory designed to clear the buffer memory when signaled by the task discriminator that a task has been completed.

17. The quantum interface control system of claim 16, further comprising an input/output port designed to allow the programming of the predefined task completion information parameter.

18. The quantum interface control system of claim 8, further comprising an input/output sub-system comprising:
an external and block input/output design in signal communication with at least one component input/output within the quantum interface control system;
an input/output communication processor in signal communication with the external and block input/output and being design to process and evaluate the characteristics of the signal from the external and block input/output; and
an input/output communication port in signal communication between the input/output processor and the external and block input/output such that the processed signals are sent to the appropriate component input/output.

19. A method of providing a quantum interaction control comprising the steps of:
producing two or more quantum signals;
manipulating the quantum signals to obtain at least one digital value characteristic of each of the quantum signals;
evaluating and modulating the quantum signals to obtain quantum form-information, the quantum form-information containing information about at least one discrete temporal and phase state that produce the greatest direct and harmonic resonance between the two or more quantum signals;
providing a feedback signal to the two or more quantum signal sources containing the quantum form-information.

20. The method of claim 19, further comprising determining a pattern match by comparing the at least one digital value signal characteristic to at least one digital pattern.

21. The method of claim 20, wherein the at least one digital pattern is stored in a memory.

22. The method of claim 20, wherein the at least one digital pattern is a user input data field.

23. The method of claim 19, wherein the step of evaluating and modulating further comprises:
determining a plurality of nodal characteristics by evaluating all resonant combinations of the two or more quantum signals;
determining a plurality of intersectional characteristics by evaluating all resonant combinations of the plurality of nodal characteristics; and
determining at least one discrete temporal and phase state by processing all resonant combinations of the plurality of intersectional characteristics.

24. The method of claim 19, further comprising forming a matrix of four or more quantum resonators; and
communicating quantum-form information between said quantum resonators by using direct quantum responsiveness and influence.

25. The method of claim 19, further comprising defining and categorizing an external signal source by measuring a quantum resonance characteristic for said external signal source.

26. The method of claim 25, wherein the information developed during the defining and categorizing step is then used to map, store, and navigate an external environment.
